(19) United States
(12) Patent Application Publication
(10) Pub. No.: US 2006/0141023 A1
(43) Pub. Date: Jun. 29, 2006

(54) Pharmaceutical compositions containing abiguanide-glitazone combination

(76) Inventors: Anupam Trehan, New Delhi (IN); Sumit Madan, New Delhi (IN); Vinod Kumar Arora, New Delhi (IN); Rajiv Malik, Vienna (AT)

Correspondence Address: RANBAXY INC., 600 COLLEGE ROAD EAST, SUITE 2100, PRINCETON, NJ, 08540, US

(21) Appl. No.: 10/534,909

(22) PCT Filed: Nov. 13, 2003
(86) PCT No.: PCT/IB03/05140

§ 371 Date: Nov. 16, 2005

(30) Foreign Application Priority Data
Nov. 15, 2002 (IN) 1155/DEL/2002

(51) Int. Cl. A61K 31/426; A61K 31/155; A61K 9/48; A61K 9/22; A61K 31/175

(52) U.S. Cl. 424/451; 424/468; 514/369; 514/592; 514/635

(57) ABSTRACT

The present invention relates to an orally administered pharmaceutical composition that is a combination of two or more antidiabetic agents in which one of the antidiabetic agents is present in an extended release form and the other antidiabetic agent is present in an immediate release form.

### FIELD OF THE INVENTION

The present invention relates to an orally administered pharmaceutical composition that is a combination of two or more antidiabetic agents in which one of the antidiabetic agents is present in an extended release form and the other antidiabetic agent is present in an immediate release form.

### BACKGROUND OF THE INVENTION

Diabetes mellitus is a metabolic disorder characterized by hyperglycemia and insulin resistance, and is often associated with other conditions such as obesity, hypertension, hyperlipidemia, cardiovascular disease, retinopathy, neuropathy, and nephropathy. The disease is progressive in nature but often can be controlled initially by diet alone, although it generally requires treatment with drugs, such as sulfonylureas, and injections of exogenous insulin. Two major forms of diabetes mellitus are now recognized: Type I and Type II. Type I diabetes, or insulin-dependent diabetes, is the result of an absolute deficiency of insulin, the hormone that regulates glucose utilization; patients with Type I diabetes are dependent on exogenous insulin for survival. Type II diabetes, or non-insulin-dependent diabetes (NIDDM), often occurs concurrent with normal, or even elevated levels of insulin, and appear to be the result of the inability of tissues to respond appropriately to insulin (i.e., insulin resistance). Insulin resistance is a major susceptibility trait of NIDDM and also is a contributing factor in arteriosclerosis, hypertension, lipid disorders and polycystic ovarian syndrome.

Conventional treatments for NIDDM have not changed substantially in many years and have significant limitations. While physical exercise and a reduction in dietary intake of calories can improve the diabetic condition, compliance with this treatment is generally poor. To increase the plasma level of insulin, physicians sometimes administer sulfonylureas (e.g., tolbutamide, glipizide). The sulfonylureas stimulate the pancreatic beta-cells to secrete more insulin. The plasma level of insulin can be directly increased by injecting insulin after the response to sulfonylureas fails and will result in insulin concentrations that stimulate even highly insulin-resistant tissues. However, dangerously low levels of plasma glucose can result from these last two treatments, and can theoretically lead to increased insulin resistance due to the even higher plasma insulin levels.

Biguanides have been the most widely used class of antidiabetics. They act by increasing insulin activity in peripheral tissues, reducing hepatic glucose output due to inhibition of gluconeogenesis, and reducing the absorption of glucose from the intestine. Metformin, phenformin, buformin, etc. belong to this group. Metformin has been widely prescribed for lowering blood glucose in patients with NIDDM and is marketed in 500, 750, 850 and 1000 mg strengths. However, because it is a short acting drug, metformin requires twice-daily or three-times-daily dosing (500-850 mg tab 2-3/day or 1000 mg bid with meals). Adverse events associated with metformin include anorexia, nausea, vomiting and diarrhea. The adverse events may be partially avoided by either reducing the initial dose and/or the maintenance dose by taking an extended-release dosage form rather than a multiple daily doses. Besides reducing the adverse events, administering an extended-release dosage form provides a reduction in the frequency of administration.

More recently, glitazones have been introduced and are widely used in the treatment of NIDDM. These agents substantially increase insulin sensitivity in muscle, liver, and adipose tissue in several NIDDM animal models, resulting in the correction of elevated plasma levels of glucose, triglycerides and nonesterified fatty acids without the occurrence of hypoglycemia. These agents, also known generically as thiazolidinediones (e.g., troglitazone, rosiglitazone and pioglitazone), function by increasing the sensitivity of peripheral tissues, such as skeletal muscle, towards insulin. Pioglitazone, the most widely used glitazone, is normally administered at doses from about 15 mg to about 45 mg, and is given as a single dose once per day. Another glitazone, rosiglitazone, is administered at doses of about 5 mg to about 10 mg per day.

Biguanides and thiazolidinediones are commercially available in the form of tablets of the individual drugs. The tablets may be in the form of either immediate release (IR) formulations or controlled release (CR) formulations and are administered orally to patients in need thereof in protocols calling for the single administration of the individual ingredient. Metformin monotherapy is used as a first line treatment in diabetic patients but may be supplemented with other drugs when the secondary failure of the therapy sets in. The addition of a thiazolidinedione agent to concurrent biguanide treatment provides a balance of stimulated release of insulin while ameliorating insulin resistance and thus provides a level of glycemic control unattainable by either medication alone.

Insulin resistance is a common feature characterizing the pathogenesis of Type II diabetes. Metformin improves glucose tolerance but cannot enhance insulin sensitivity. In contrast, glitazones improve glycemic control by improving insulin sensitivity. The glitazones are highly selective and potent agonists for the peroxisome proliferator-activated receptor-gamma (PPAR-γ). Activation of PPAR-γ nuclear receptors regulates the transcription of insulin responsive genes involved in the control of glucose production, transport, and utilization. In addition, PPAR-γ-responsive genes also participate in the regulation of fatty acid metabolism. The antidiabetic activity of glitazones has been demonstrated in those Type II diabetes in which hyperglycemia and/or impaired glucose tolerance is a consequence of insulin resistance in target tissues. A single administration of glitazones activates the insulin receptors for an extended period and may thus be administered as a single dose without there being a need to maintain the plasma concentration.

A combination therapy of a biguanide and a glitazone, therefore, has a synergistic effect on glucose control because both agents act by different but complementary mechanisms. Clinical evaluations have demonstrated the method of treating diabetes by employing combinations of biguanides and glitazones (WO 00/27401 and U.S. Pat. No. 6,011,049). Moreover, pharmaceutical compositions having combinations of biguanides and thiazolidinediones and providing controlled or immediate release of both of the drugs are known in the art. For example, U.S. Pat. No. 6,296,874 and published U.S. patent application Ser. Nos. 20010036478 A1, 2010034374 A1, and 20010046545 A1 (Adjei et al.) describe controlled release core combinations of a glitazone with a biguanide chosen from metformin, phenformin or buformin in a single dosage form. The patent applications of Adjei et al. disclose the preparation of such combinations using either silicate polymers or polysaccharides.

U.S. Pat. Nos. 6,166,043 and 6,172,090 disclose methods for reducing the amounts and side effects of active components administered to a diabetic patient. One method disclosed includes administering a therapeutically effective amount of an insulin sensitivity enhancer in combination with a biguanide. The insulin sensitivity enhancer in the system is a thiazolidinedione selected from pioglitazone and troglitazone, while the biguanide is selected from metformin, phenformin and buformin. The combination may be administered as an admixture of the agents or the agents administered independently. The thiazolidinedione and the biguanide may be in the form of a conventional immediate release composition.

Although combinations of two antidiabetic agents are known in the art and are convenient to formulate, a combination providing extended release of a water-soluble active ingredient, e.g., a biguanide, and immediate release of a water-insoluble or sparingly soluble active, e.g., a glitazone, is difficult to achieve.

### SUMMARY OF THE INVENTION

In one general aspect there is provided a solid pharmaceutical dosage form for oral administration. The dosage form includes an extended release layer that includes a biguanide and an immediate release layer that includes a glitazone.

Embodiments of the dosage form may include one or more of the following features. For example, the biguanide may be one or more of metformin, phenformin, and buformin. The glitazone may be one or more of pioglitazone, rosiglitazone, troglitazone, ciglitazone and englitazone. After oral administration the biguanide may be released over a period of about 4 to about 36 hours and, more particularly, over a period of about 8 to about 24 hours.

The dosage form may be tablets or capsules. The tablet may include a coating. The capsules may include one or more of pellets, beads, granules, multiparticulates, tablets and powder.

The extended release layer may be a matrix and the matrix may have a uniform mixture of the biguanide and one or more rate controlling polymers. The one or more rate-controlling polymers may be hydrophilic polymers, hydrophobic polymers, or a combination thereof. The matrix may further include one or more pharmaceutically acceptable excipients. The pharmaceutically acceptable excipients may be one or more of diluents, lubricants, disintegrants, binders, glidants, coloring and flavoring agents.

The biguanide may layered onto a pharmaceutically inert core or seed. The inert core or seed may be hydrosoluble or hydroinsoluble.

The immediate release outer layer may further include film-forming polymers and optionally other pharmaceutically acceptable excipients. The film-forming polymers may be water-soluble polymers. The pharmaceutically acceptable excipients may be one or more of plasticizers, opacifiers and colorants.

The dosage form may further include a wetting agent in the immediate release layer such that the immediate release layer includes a glitazone and the wetting agent in a weight ratio ranging from about 10:1 to about 1:25. The wetting agent may be selected from amongst hydrophilic and hydrophobic surfactants. The hydrophilic surfactants may be selected from one or more of non-ionic surfactants, ionic surfactants or mixtures thereof.

The hydrophobic surfactants may be selected from one or more of alcohols; polyoxyethylene alkylethers; fatty acids; glycerol fatty acid monoesters; glycerol fatty acid diesters; acetylated glycerol fatty acid monoesters; acetylated glycerol fatty acid diesters, lower alcohol fatty acid esters; polyethylene glycol fatty acid esters; polyethylene glycol glycerol fatty acid esters; polypropylene glycol fatty acid esters; polyoxyethylene glycerides; lactic acid derivatives of monoglycerides; lactic acid derivatives of diglycerides; propylene glycol diglycerides; sorbitan fatty acid esters; polyoxyethylene sorbitan fatty acid esters; polyoxyethylene-polyoxypropylene block copolymers, polyethyleneglycols as esters or ethers, polyethoxylated castor oil; polyethoxylated hydrogenated castor oil, polyethoxylated fatty acid from castor oil or polyethoxylated fatty acid from castor oil or polyethoxylated fatty acid from hydrogenated castor oil.

The non-ionic surfactants may be selected from one or more of alkylglucosides; alkylmaltosides; alkylthioglucosides; lauryl macrogolglycerides; caprylocaproyl macrogolglycerides, polyoxyethylene alkyl ethers; polyoxyethylene alkylphenols; polyethylene glycol fatty acid esters; polyethylene glycol glycerol fatty acid esters; polyoxyethylene sorbitan fatty acid esters; polyoxyethylene-polyoxypropylene block copolymers; polyglycerol fatty acid esters; polyoxyethylene glycerides; polyoxyethylene sterols, derivatives, and analogues thereof; polyoxyethylene vegetable oils; polyoxyethylene hydrogenated vegetable oils; reaction products of polyols and at least one member of the group consisting of fatty acids, glycerides, vegetable oils, hydrogenated vegetable oils, and sterols; sugar esters, sugar ethers; sucroglycerides; and mixtures thereof.

The ionic surfactants may be selected from one or more of alkyl ammonium salts; bile acids and salts, analogues, and derivatives thereof; fatty acid derivatives of amino acids, oligopeptides, and polypeptides; glyceride derivatives of amino acids, oligopeptides, and polypeptides; acyl lactylates; monoacetylated tartaric acid esters of monoglycerides, monoacetylated tartaric acid esters of diglycerides, diacetylated tartaric acid esters of monoglycerides, diacetylated tartaric acid esters of diglycerides; succinylated monoglycerides; citric acid esters of monoglycerides; citric acid esters of diglycerides; alginate salts; propylene glycol alginate; lecithins and hydrogenated lecithins; lysolecithin and hydrogenated lysolecithins; lysophospholipids and derivatives thereof; phospholipids and derivatives thereof; salts of alkylsulfates; salts of fatty acids; sodium docusate; and mixtures thereof.

The extended release layer may be a core and the immediate release layer may cover at least a portion of the core. The dosage form may be a bilayered dosage form. The dosage form may further include one or more of sulfonylurea, insulin, alpha-glucosidase inhibitors, meglitinides, fibrates, statins, squalene synthesis inhibitors and angiotensin-converting enzyme inhibitors.

In another general aspect there is provided a process for preparing a solid, orally administered pharmaceutical dosage form of an extended release core of a biguanide and an immediate release layer of a glitazone. The process includes (a.) dispersing the biguanide in a solid matrix to form a core having a surface; and (b.) layering the immediate release layer of a glitazone on the surface of the core.

Embodiments of the process may include one or more of the following features. For example, layering the immediate release layer may further include layering one or more wetting agents. The glitazone and the one or more wetting agents may be present in the immediate release layer in a weight ratio ranging from about 10:1 to about 1:25. The one or more wetting agents may be selected from amongst hydrophilic or hydrophobic surfactants. The hydrophilic surfactants may be selected from one or more of non-ionic surfactants, ionic surfactants or mixtures thereof.

The hydrophobic surfactants may be selected from one or more of alcohols; polyoxyethylene alkylethers; fatty acids; glycerol fatty acid monoesters; glycerol fatty acid diesters; acetylated glycerol fatty acid monoesters; acetylated glycerol fatty acid diesters, lower alcohol fatty acid esters; polyethylene glycol fatty acid esters; polyethylene glycol glycerol fatty acid esters; polypropylene glycol fatty acid esters; polyoxyethylene glycerides; lactic acid derivatives of monoglycerides; lactic acid derivatives of diglycerides; propylene glycol diglycerides; sorbitan fatty acid esters; polyoxyethylene sorbitan fatty acid esters; polyoxyethylene-polyoxypropylene block copolymers, polyethyleneglycols as esters or ethers, polyethoxylated castor oil; polyethoxylated hydrogenated castor oil, polyethoxylated fatty acid from castor oil or polyethoxylated fatty acid from castor oil or polyethoxylated fatty acid from hydrogenated castor oil.

The non-ionic surfactants may be selected from one or more of alkylglucosides; alkylmaltosides; alkylthioglucosides; lauryl macrogolglycerides; caprylocaproyl macrogolglycerides, polyoxyethylene alkyl ethers; polyoxyethylene alkylphenols; polyethylene glycol fatty acid esters; polyethylene glycol glycerol fatty acid esters; polyoxyethylene sorbitan fatty acid esters; polyoxyethylene-polyoxypropylene block copolymers; polyglycerol fatty acid esters; polyoxyethylene glycerides; polyoxyethylene sterols, derivatives, and analogues thereof; polyoxyethylene vegetable oils; polyoxyethylene hydrogenated vegetable oils; reaction products of polyols and at least one member of the group consisting of fatty acids, glycerides, vegetable oils, hydrogenated vegetable oils, and sterols; sugar esters, sugar ethers; sucroglycerides; and mixtures thereof.

The ionic surfactants may be selected from amongst alkyl ammonium salts; bile acids and salts, analogues, and derivatives thereof; fatty acid derivatives of amino acids, oligopeptides, and polypeptides; glyceride derivatives of amino acids, oligopeptides, and polypeptides; acyl lactylates; monoacetylated tartaric acid esters of monoglycerides, monoacetylated tartaric acid esters of diglycerides, diacetylated tartaric acid esters of monoglycerides, diacetylated tartaric acid esters of diglycerides; succinylated monoglycerides; citric acid esters of monoglycerides; citric acid esters of diglycerides; alginate salts; propylene glycol alginate; lecithins and hydrogenated lecithins; lysolecithin and hydrogenated lysolecithins; lysophospholipids and derivatives thereof; phospholipids and derivatives thereof; salts of alkylsulfates; salts of fatty acids; sodium docusate; and mixtures thereof

The biguanide may be selected from one or more of metformin, phenformin and buformin. The glitazone may be selected from one or more of pioglitazone, rosiglitazone, troglitazone, ciglitazone and englitazone. After oral administration the biguanide may be released over a period of about 4 to about 36 hours and, more particularly, over a period of about 8 to about 24 hours.

The process may further include forming a tablet or a capsule. The process may still further include coating the tablet. The capsule may contain one or more of pellets, beads, granules, multiparticulates, tablets and powder.

The core may be a matrix and the matrix may be a uniform mixture of the biguanide and one or more rate controlling polymers. The one or more rate-controlling polymers may be either or both of hydrophilic and hydrophobic. The matrix may further include one or more pharmaceutically acceptable excipients. The pharmaceutically acceptable excipients may include one or more of diluents, lubricants, disintegrants, binders, glidants, colorants, and flavorants.

The biguanide may be layered onto pharmaceutically inert core or seeds. The inert core or seeds may be hydrosoluble or hydroinsoluble.

The immediate release outer layer may further include film-forming polymers and optionally other pharmaceutically acceptable excipients. The film-forming polymers may be water-soluble polymers. The pharmaceutically acceptable excipients may be one or more of plasticizers, opacifiers and colorants.

The process may further include placing a seal-coat over the core, the seal-coat including hydrophilic polymers.

In another general aspect there is provided a process for preparing a bilayered, solid, orally administered pharmaceutical dosage form of a biguanide and a glitazone. The process includes (a.) dispersing the biguanide in an extended release carrier base material; (b.) separately dispersing the glitazone in an immediate release carrier base material; and (c.) compressing the material of step a and step b to form bilayered tablet.

Embodiments of the process may include one or more of the following features. For example, the immediate release carrier base material may further include one or more wetting agents before or after dispersing the glitazone. The glitazone and the one or more wetting agents may be present in a weight ratio ranging from about 10:1 to about 1:25. The one or more wetting agents may be selected from amongst hydrophilic or hydrophobic surfactants. The hydrophilic surfactants may be selected from one or more of non-ionic surfactants, ionic surfactants or mixtures thereof.

The hydrophobic surfactants may be selected from one or more of alcohols; polyoxyethylene alkylethers; fatty acids; glycerol fatty acid monoesters; glycerol fatty acid diesters; acetylated glycerol fatty acid monoesters; acetylated glycerol fatty acid diesters, lower alcohol fatty acid esters; polyethylene glycol fatty acid esters; polyethylene glycol glycerol fatty acid esters; polypropylene glycol fatty acid esters; polyoxyethylene glycerides; lactic acid derivatives of monoglycerides; lactic acid derivatives of diglycerides; propylene glycol diglycerides; sorbitan fatty acid esters; polyoxyethylene sorbitan fatty acid esters; polyoxyethylene-polyoxypropylene block copolymers, polyethyleneglycols as esters or ethers, polyethoxylated castor oil; polyethoxylated hydrogenated castor oil, polyethoxylated fatty acid from castor oil or polyethoxylated fatty acid from castor oil or polyethoxylated fatty acid from hydrogenated castor oil.

The non-ionic surfactants may be selected from one or more of alkylglucosides; alkylmaltosides; alkylthioglucosides; lauryl macrogolglycerides; caprylocaproyl macrogolglycerides, polyoxyethylene alkyl ethers; polyoxyethylene alkylphenols; polyethylene glycol fatty acid esters; polyethylene glycol glycerol fatty acid esters; polyoxyethylene sorbitan fatty acid esters; polyoxyethylene-polyoxypropylene block copolymers; polyglycerol fatty acid esters; polyoxyethylene glycerides; polyoxyethylene sterols, derivatives, and analogues thereof; polyoxyethylene vegetable oils; polyoxyethylene hydrogenated vegetable oils; reaction products of polyols and at least one member of the group consisting of fatty acids, glycerides, vegetable oils, hydrogenated vegetable oils, and sterols; sugar esters, sugar ethers; sucroglycerides; and mixtures thereof.

The ionic surfactants may be selected from amongst alkyl ammonium salts; bile acids and salts, analogues, and derivatives thereof; fatty acid derivatives of amino acids, oligopeptides, and polypeptides; glyceride derivatives of amino acids, oligopeptides, and polypeptides; acyl lactylates; monoacetylated tartaric acid esters of monoglycerides, monoacetylated tartaric acid esters of diglycerides, diacetylated tartaric acid esters of mono glycerides, diacetylated tartaric acid esters of diglycerides; succinylated monoglycerides; citric acid esters of monoglycerides; citric acid esters of diglycerides; alginate salts; propylene glycol alginate; lecithins and hydrogenated lecithins; lysolecithin and hydrogenated lysolecithins; lysophospholipids and derivatives thereof; phospholipids and derivatives thereof; salts of alkylsulfates; salts of fatty acids; sodium docusate; and mixtures thereof.

The biguanide may be selected from one or more of metformin, phenformin and buformin. The glitazone may be selected from one or more of pioglitazone, rosiglitazone, troglitazone, ciglitazone and englitazone.

After oral administration, the biguanide is released over a period of about 4 to about 36 hours and, more particularly, over a period of about 8 to about 24 hours.

The process may further include forming a tablet or a capsule. The process may still further include coating the tablet. The capsule may contain one or more of pellets, beads, granules, multiparticulates, tablets and powder.

The biguanide layer may be a matrix and the matrix may be a uniform mixture of the biguanide and one or more rate controlling polymers. The one or more rate-controlling polymers may be either or both of hydrophilic polymers and hydrophobic polymers. The matrix may further include one or more pharmaceutically acceptable excipients. The pharmaceutically acceptable excipients may be one or more of diluents, lubricants, disintegrants, binders, glidants, colorants, and flavorants. The biguanide may be layered onto pharmaceutically inert core or seeds. The inert core or seeds may be hydrosoluble or hydroinsoluble.

The immediate release carrier base material may further include one or more film-forming polymers and optionally other pharmaceutically acceptable excipients. The film-forming polymers may be water-soluble polymers. The pharmaceutically acceptable excipients may be one or more of plasticizers, opacifiers and colorants.

The process may further include providing a seal-coat of one or more hydrophilic polymers between the two layers.

In another general aspect there is provided a method of treating non-insulin dependent diabetes mellitus in a patient in need thereof. The method includes administering a solid, pharmaceutical dosage form of the combination of a biguanide and a glitazone. The dosage form provides an extended-release of the biguanide and an immediate release of the glitazone.

Embodiments of the method may include one or more of the following features or those described above. For example, the biguanide may be one or more of metformin, phenformin, and buformin and, in particular, may be metformin. The glitazone may be one or more of pioglitazone, rosiglitazone, troglitazone, ciglitazone and englitazone and, in particular, may be pioglitazone.

After oral administration of the dosage form, the biguanide is released over a period of about 4 to about 36 hours and, more particularly, over a period of about 8 to about 24 hours. The dosage form may be tablets or capsules. The dosage form may further include one or more of sulfonylurea, insulin, alpha-glucosidase inhibitors, meglitinides, fibrates, statins, squalene synthesis inhibitors and angiotensin-converting enzyme inhibitors.

The details of one or more embodiments of the inventions are set forth in the description below. Other features, objects and advantages of the inventions will be apparent from the description and claims.

### DETAILED DESCRIPTION OF THE INVENTION

Hydrophobic therapeutic agents, i.e., therapeutic compounds having poor solubility in aqueous solution, are difficult to formulate as a dosage form that can be effectively administered to patients. A well-designed formulation must, at a minimum, be capable of presenting a therapeutically effective amount of the hydrophobic compound to the desired absorption site, in an absorbable form. Even this minimal functionality is difficult to achieve when delivery of the hydrophobic therapeutic agent requires interaction with aqueous physiological environments, such as gastric and intestinal fluids. Pharmaceutical compositions for delivering such hydrophobic therapeutic agents must carry the hydrophobic compound through the aqueous environment while both maintaining the hydrophobic compound in an absorbable form and avoiding the use of physiologically harmful solvents or excipients.

A similar problem is faced when formulating extended release dosage forms of highly soluble therapeutic agents. The high solubility of the therapeutic agent requires the incorporation of a high percentage of the polymer to achieve a desired release profile and prolonged effect. Further, an additional constraint is the necessity of controlling the initial burst of the drug from the formulation.

Therefore, there remains a need for pharmaceutical compositions for oral administration that include a combination of one or more hydrophobic, water-insoluble therapeutic agents, i.e., a glitazone, in immediate release form, and a highly water-soluble therapeutic agent, i.e., a biguanide, in an extended-release form with the characteristics of achieving an effect over 24 hours after once daily administration.

It has now been discovered that a dosage form can be prepared that includes: (a) one layer or a core from which a single highly water-soluble active ingredient is released on a prolonged basis and (b) a coating or layer from which another active ingredient is released on an immediate-release basis. The dosage form provides a high degree of uniformity in the immediate-release portion, even in those circumstances in which the drug in the immediate-release portion is either insoluble or only sparingly soluble in water. This result is achieved by incorporating one or more wetting agents in the immediate release layer in an amount in which the weight ratio of the glitazone to wetting agent ranges from 10:1 to about 1:25.

Specifically, in one aspect there is provided a dosage form that contains both a glitazone and a biguanide. The glitazone is contained in an immediate-release form so that it is released substantially immediately upon ingestion (i.e., upon swallowing). Generally at least 80% of the glitazone is released from the dosage form within an hour after administration. The biguanide, by contrast, releases in a sustained fashion; at least about 75% of the drug contained in the dosage form is released over a period of 4 to 36 hours, preferably about 8 to 24 hours. The term “about” as used above and elsewhere herein means plus or minus 10% for each of the numerical limits.

The pharmaceutical compositions of the present invention can be administered orally in the form of tablets, such as coated-tablets, bilayered tablets or multi-layered tablets, or in form of capsules containing pellets, beads, granules, multiparticulates, tablets or powder.

Biguanide as employed herein is intended to include metformin, phenformin and buformin including their salts, solvates, hydrates and polymorphs. Particularly, the biguanide used may be metformin. Different salts of metformin that can be used include hydrochloride, acetate, maleate, fumarate, succinate and other salts. The daily effective dose of metformin may range from about 500 mg to about 2550 mg, and, in particular, the dose may be a single dose of about 500 mg to about 1000 mg. The biguanide is present in an amount from about 40% to about 75% by weight of the total composition.

The biguanide may be incorporated in an extended release carrier base by dispersing the biguanide in a rate-controlling polymer matrix, as described in our pending application, published as WO 03/028704. Alternatively, the biguanide may be layered onto pharmaceutically acceptable inert cores or seeds in admixture with rate-controlling polymers or surrounded by rate-controlling polymers.

The term matrix, as used herein, refers to a uniform mixture of a biguanide, rate-controlling polymers, and, optionally, other pharmaceutically acceptable excipients. The rate-controlling polymers may be hydrophilic, hydrophobic or a combination thereof. The rate-controlling polymers are uniformly dispersed throughout the matrix to achieve uniform drug release. Hydrophilic polymers of the present invention include, for example, cellulose derivatives such as hydroxypropylcellulose, hydroxypropyl methylcellulose, hydroxyethylcellulose, hydroxymethylcellulose, carboxymethylcellulose, methylcellulose, sodium carboxymethylcellulose or combinations thereof. The hydrophobic polymers include one or more of poly(ethylene)oxide, ethyl cellulose, cellulose acetate, cellulose acetate butyrate, hydroxypropyl methylcellulose phthalate, poly(alkyl)methacrylate, copolymers of acrylic or methacrylic acid esters, waxes, shellac, and hydrogenated vegetable oils.

In addition to the one or more active ingredients and rate-controlling polymers, the matrix may contain other pharmaceutically acceptable excipients that act in one or more capacities as diluents, binders, lubricants, glidants, colorants or flavoring agents.

Suitable diluents include pharmaceutically acceptable inert fillers such as microcrystalline cellulose, lactose, dibasic calcium phosphate, mannitol, starch, sorbitol, sucrose, dextrose, maltodextrin or mixtures thereof

Suitable binders include one or more of polyvinyl pyrrolidone, lactose, starches, gums, waxes, gelatin, polymers or mixtures thereof.

Suitable lubricants include one or more of colloidal silicon dioxide, talc, stearic acid, magnesium stearate, magnesium silicate, polyethylene, sodium benzoate, sodium lauryl sulphate, fumaric acid, zinc stearate, paraffin, or mixtures thereof.

Suitable glidants include one or more of talc and colloidal silicon dioxide.

The matrix may be made by any pharmaceutically acceptable technique that achieves uniform blending, e.g., dry blending, dry granulation, wet granulation, compaction, and fluid bed granulation.

The matrix formed can be compressed to form the tablets. Alternatively, the matrix may be formulated as a plurality of discrete or aggregated particles, pellets, beads or granules.

As described above, the biguanide may be layered onto cores or seeds. The inert core or seeds may be hydro soluble, such as sucrose, lactose, maltodextrin and the like, or hydro insoluble, such as microcrystalline cellulose, partially pregelatinized starch, dicalcium phosphate and the like. The biguanide and the rate controlling polymer can be coated as single layer or as separate layers on these inert cores, granulated with the inert cores, or mixed with the inert cores and extruded and spheronized to form the pellets.

The coating may be applied to the inert/active core using a conventional coating pan, a spray coater, a rotating perforated pan, or an automated system, such as a centrifugal fluidizing (CF) granulator, a fluidized bed process, or any other suitably automated coating equipment.

The extended-release core containing a biguanide may optionally be coated to seal the core. The coated active cores may be dried under conditions effective for drying, e.g., in an oven or by means of gas in a fluidized bed.

Finally, these beads/pellets may be filled into capsules or compressed to form the tablets. The capsule dosage form may include a plurality of pellets, granules or beads or a single compressed tablet which release the biguanide over an extended period of time.

Glitazone as employed herein is intended to include, but is not limited to, pioglitazone, rosiglitazone, troglitazone, ciglitazone, englitazone, and their salts, solvates, hydrates and polymorphs. In particular, the glitazone may be pioglitazone. The daily effective dose of pioglitazone may range from 5 mg to 50 mg and, in particular, the dose may be a single dose of 10 mg to 45 mg. The glitazone may be present in an amount of from about 0.5% to about 10% by weight of the total composition.

A glitazone can be incorporated into the dosage form as an immediate release component in a variety of ways. For example, it can be incorporated into an exterior coating of a tablet from which it releases substantially immediately upon ingestion. Such a coating can similarly be applied to each of the particles that make up a multiparticulate system, i.e., granules, beads. If the dosage form is to be a capsule, the glitazone can be contained in a single pellet inside the capsule from which it releases substantially immediately once the capsule shell dissolves. Alternatively, the glitazone can be contained in several smaller pellets, can be present as one or more immediate release particles, or can be present as one or more immediate release layers over the extended release cores or beads. Any conventional method may be used for the preparation of the layer of the glitazone. Conventional pharmaceutically acceptable excipients may be incorporated into this layer. These excipients may include diluents, binders and lubricants.

The coating composition for coating the glitazone may include water-soluble polymers such as polyvinyl pyrrolidine, hydroxypropyl cellulose, polyvinyl alcohol, hydroxypropyl methylcellulose and the like. The polymer may be applied as a solution in an organic solvent or as an aqueous dispersion. The solvent may be, for example, one or more of water; alcohol such as ethyl alcohol or isopropyl alcohol; ketones such as acetone or ethylmethyl ketone; and chlorinated hydrocarbons such as dichloroethane and trichloroethane. The coating composition may also include plasticizers, opacifiers and colorants. Any conventional coating equipment may be employed to facilitate coating, including a centrifugal fluidized bed coating apparatus, pan coating apparatus, or fluidized bed granulating coating apparatus.

Due to poor dispersibility in solvents, the film-coating composition that includes the glitazone may optionally include a wetting agent. Suitable wetting agents include hydrophilic and hydrophobic surfactants. Hydrophilic surfactants may be selected from one or more of hydrophilic non-ionic surfactants, hydrophilic ionic surfactants, and combinations thereof.

Non-ionic surfactants may be selected from one or more of alkylglucosides; alkylmaltosides; alkylthioglucosides; lauryl macrogolglycerides; caprylocaproyl macrogolglycerides, polyoxyethylene alkyl ethers; polyoxyethylene alkylphenols; polyethylene glycol fatty acid esters; polyethylene glycol glycerol fatty acid esters; polyoxyethylene sorbitan fatty acid esters; polyoxyethylene-polyoxypropylene block copolymers; polyglycerol fatty acid esters; polyoxyethylene glycerides; polyoxyethylene sterols, derivatives, and analogues thereof; polyoxyethylene vegetable oils; polyoxyethylene hydrogenated vegetable oils; reaction products of polyols and at least one member of the group consisting of fatty acids, glycerides, vegetable oils, hydrogenated vegetable oils, and sterols; sugar esters, sugar ethers; sucroglycerides; and mixtures thereof.

Ionic surfactants may be selected from one or more of alkyl ammonium salts; bile acids and salts, analogues, and derivatives thereof; fatty acid derivatives of amino acids, oligopeptides, and polypeptides; glyceride derivatives of amino acids, oligopeptides, and polypeptides; acyl lactylates; monoacetylated tartaric acid esters of monoglycerides, monoacetylated tartaric acid esters of diglycerides, diacetylated tartaric acid esters of monoglycerides, diacetylated tartaric acid esters of diglycerides; succinylated monoglycerides; citric acid esters of monoglycerides; citric acid esters of diglycerides; lysolecithin and hydrogenated lysolecithins; lysophospholipids and derivatives thereof; phospholipids and derivatives thereof; salts of alkylsulfates; salts of fatty acids; sodium docusate; and mixtures thereof.

Hydrophobic surfactants may be selected from one or more of alcohols; polyoxyethylene alkylethers; fatty acids; glycerol fatty acid monoesters; glycerol fatty acid diesters; acetylated glycerol fatty acid monoesters; acetylated glycerol fatty acid diesters, lower alcohol fatty acid esters; polyethylene glycol fatty acid esters; polyethylene glycol glycerol fatty acid esters; polypropylene glycol fatty acid esters; polyoxyethylene glycerides; lactic acid derivatives of monoglycerides; lactic acid derivatives of diglycerides; propylene glycol diglycerides; sorbitan fatty acid esters; polyoxyethylene sorbitan fatty acid esters; polyoxyethylene-polyoxypropylene block copolymers, polyethyleneglycols as esters or ethers, polyethoxylated castor oil; polyethoxylated hydrogenated castor oil, polyethoxylated fatty acid from castor oil or polyethoxylated fatty acid from castor oil or polyethoxylated fatty acid from hydrogenated castor oil.

The glitazone and the wetting agent may be present in a weight ratio ranging from about 10:1 to about 1:25.

Of one of the embodiments, there is provided a bilayered dosage form for the combination of a biguanide and a glitazone. The term ‘bilayered’ as used herein is meant to encompass solid dosage forms in which there are two separate drug layers, with only one surface in contact with each other. These may be prepared, for example, by compressing additional granulation on a previously compressed granulation or alternatively by feeding previously compressed tablets into a machine and compressing another granulation layer around the preformed tablets.

An example of a bi-layer tablet manufacturing method includes: (1) blending a quantity of a glitazone with various excipients, colorants, and/or other pharmaceutically acceptable excipients and additives to form an immediate release formulation, (2) blending a quantity of a biguanide with a rate-controlling polymer, and various excipients, colorants, and/or other pharmaceutical additives to form an extended release formulation, and (3) compressing a quantity of the immediate release formulation of the glitazone with a quantity of the extended release formulation of the biguanide to form a bi-layer tablet.

One of the embodiments includes providing a seal coat of hydrophilic polymers between the extended-release and immediate-release layers.

Other embodiments include modifications relating to coating the tablet with the polymer in order to modify the release of the drug. The solid dosage forms may be optionally coated with non-functional coatings well known in the art, or with coatings that further modify the release of the drug from the dosage form. All such modifications as may be done and understood by those who are skilled in the art are within the scope of the present invention. For example, one such modification includes making the composition into a layered tablet in which the composition provides extended release of more than one therapeutic agent, or extended release of one of the therapeutic agents and immediate or delayed release of the other therapeutic agent(s).

### EXAMPLE 1

| | INGREDIENTS | Mg/tablet |
|---|---|---|
| CORE | Metformin Hydrochloride | 500 |
| | Microcrystalline Cellulose | 245 |
| | Sodium Carboxymethyl Cellulose | 150 |
| | Purified Water | q.s. |
| | Hydroxypropyl methylcellulose | 100 |
| | Magnesium Stearate | 5 |
| SEAL | Hydroxypropyl methylcellulose E5 | 15.6 |
| COAT | Polyethylene glycol 4000 | 4.8 |
| | Titanium Dioxide | 2.4 |
| | Talc | 1.2 |
| | Purified Water | q.s. |
| ACTIVE | Pioglitazone Hydrochloride equivalent | 39.672 |
| COAT | to Pioglitazone (30 mg) | |
| | Caprylocaporyl Macrogolglycerides | 18 |
| | Hydroxypropyl methylcellulose E5 | 40.3 |
| | Polyethylene glycol 4000 | 12.4 |
| | Titanium Dioxide | 6.2 |
| | Talc | 3.1 |
| | Purified Water | q.s. |

Procedure:

1. Metformin hydrochloride was milled through a 1 mm screen and mixed with microcrystalline cellulose and sodium carboxymethyl cellulose. The blend was sieved through a No. 44 mesh, transferred to a rapid mixer granulator, and wet granulated with purified water. The granules were dried in a fluid bed dryer, sized through a multimill, and sifted through a No. 30 mesh.

2. Hydroxypropyl methylcellulose was separately sifted through a No. 30 mesh and mixed with the granules of step 1 in a low shear mixer. The blend was then mixed with magnesium stearate and compressed into tablets.

3. A coating dispersion was prepared by dispersing all of the ingredients of the seal coat in water. The tablets were coated with this dispersion until a weight build up of 2% was achieved.

4. To prepare the active coat, caprylocaproyl monoglyceride was dissolved in purified water. To this solution, pioglitazone hydrochloride was added with stirring to form a dispersion. The other ingredients of the active coat were added with stirring to this dispersion and the resulting dispersion was then spray coated upon the tablets obtained from step 3 until a weight build up of 10% was achieved.

### EXAMPLE 2

| | INGREDIENTS | Mg/tablet |
|---|---|---|
| CORE | Metformin Hydrochloride | 500 |
| | Microcrystalline Cellulose | 245 |
| | Sodium Carboxymethyl Cellulose | 150 |
| | Hydroxypropyl methylcellulose | 100 |
| | Magnesium Stearate | 5 |
| SEAL | Hydroxypropyl methylcellulose E5 | 15.6 |
| COAT | Polyethylene glycol 4000 | 4.8 |
| | Titanium Dioxide | 2.4 |
| | Talc | 1.2 |
| | Purified Water | q.s. |
| ACTIVE | Pioglitazone Hydrochloride equivalent | 39.672 |
| COAT | to Pioglitazone (30 mg) | |
| | Hydroxypropyl methylcellulose E5 | 37.2 |
| | Polyethylene glycol 400 | 7.2 |
| | Titanium Dioxide | 6.2 |
| | Talc | 12.0 |
| | Methylene chloride | q.s. |
| | Isopropyl alcohol | q.s. |

Procedure:

1. Metformin hydrochloride was milled through a 1 mm screen and mixed with microcrystalline cellulose and sodium carboxymethyl cellulose. The blend was sieved through a No. 44 mesh.

2. Hydroxypropyl methylcellulose was separately sifted through a No. 30 mesh and mixed with the blend of step 1 in a low shear mixer. The blend was then mixed with magnesium stearate compressed into tablets.

3. A coating dispersion was prepared by dispersing all of the ingredients of the seal coat in water. The tablets were coated with this dispersion until a weight build up of 2% was achieved.

4. To prepare the active coat, pioglitazone hydrochloride was dissolved in a methylene chloride and isopropyl alcohol mix (ratio of 2:1). The other ingredients of the active coat then were added with stirring to this solution and the resulting dispersion was spray coated upon the tablets obtained from step 3 until a weight build up of 10% was achieved.

### EXAMPLE 3

| | INGREDIENTS | Mg/tablet |
|---|---|---|
| CORE | Metformin Hydrochloride | 500 |
| | Microcrystalline Cellulose | 245 |
| | Sodium Carboxymethyl Cellulose | 150 |
| | Hydroxypropyl methylcellulose | 100 |
| | Magnesium Stearate | 5 |
| SEAL | Hydroxypropyl methylcellulose E5 | 15.6 |
| COAT | Polyethylene glycol 4000 | 4.8 |
| | Titanium Dioxide | 2.4 |
| | Talc | 1.2 |
| | Purified Water | q.s. |
| ACTIVE | Pioglitazone Hydrochloride equivalent | 19.836 |
| COAT | to Pioglitazone (15 mg) | |
| | Caprylocaporyl Macrogolglycerides | 14.4 |
| | Hydroxypropyl methylcellulose E5 | 40.3 |
| | Polyethylene glycol 4000 | 12.4 |
| | Titanium Dioxide | 6.2 |
| | Talc | 3.1 |
| | Purified Water | q.s. |

Procedure:

1. Metformin hydrochloride was milled through a 1 mm screen and mixed with microcrystalline cellulose and sodium carboxymethyl cellulose. The blend was sieved through a No. 44 mesh.

2. Hydroxypropyl methylcellulose was separately sifted through a No. 30 mesh and mixed with the blend of step 1 in a low shear mixer. The blend was then mixed with magnesium stearate, passed through a roller compactor, and milled again to form granules. These granules were then compressed into tablets.

3. A coating dispersion was prepared by dispersing all of the ingredients of the seal coat in water. The tablets were coated with this dispersion until a weight build up of 2% was achieved.

4. To prepare the active coat, caprylocaproyl monoglyceride was dissolved in purified water. To this solution, pioglitazone hydrochloride was added with stirring to form dispersion. The other ingredients of the active coat were added with stirring to this dispersion and the resulting dispersion was spray coated upon the tablets obtained from step 3 until the weight build up of 8.0% was achieved.

A comparative dissolution profile of metformin hydrochloride in the innovator's marketed tablets (Glucophage XR 500 mg) and tablet formulation made in accordance with the invention disclosed in Example 3 was obtained. The dissolution was carried out in USP Apparatus Type I (basket) at a speed of 100 rpm. The medium was 900 ml phosphate buffer pH 6.8. The data obtained is disclosed in Table 1.

TABLE 1
Comparative dissolution profile of metformin hydrochloride in
Glucophage XR 500 mg versus the tablets of Example 3

| | |
|---|---|
| | Percent (%) |
| Time | Metformin hydrochloride released |

| (hrs) | Glucophage XR | Tablets (Example 3) |
|---|---|---|
| 0 | 0 | 0 |
| 1 | 29 | 28 |
| 2 | 41 | 43 |
| 4 | 60 | 65 |
| 8 | 83 | 92 |
| 10 | 90 | 100 |
| 12 | 99 | 101 |

From the results, it is evident that approximately all the drug is released in twelve hours in both formulations thereby showing substantially similar dissolution profiles.

A comparative dissolution profile of pioglitazone hydrochloride in the innovator's marketed tablets (Actos, 15 mg) and tablet formulation made in accordance with Example 3 was obtained. The dissolution was carried out in USP Apparatus Type I at a speed of 100 rpm. The medium was 900 ml 0.1 N hydrochloric acid. The data obtained is disclosed in Table 2.

TABLE 2
Comparative dissolution profile of pioglitazone hydrochloride in
Actos 15 mg versus tablets of Example 3

| | |
|---|---|
| | Percent (%) |
| Time | Pioglitazone hydrochloride released |

| (hrs) | Actos 15 mg | Tablets (Example 3) |
|---|---|---|
| 0 | 0 | 0 |
| 15 | 100 | 95 |
| 30 | 101 | 104 |
| 45 | 101 | 106 |

From the results, it is evident that more than 95% of the drug is released in 15 minutes in both formulations thereby showing substantially similar dissolution profiles.

Pharmacokinetics

The drug release was evaluated in vivo in a randomized, two treatment, two period, single dose, crossover bioavailability study. The study was conducted in twelve healthy, adult, male, human subjects under fasting conditions. A single OD dose of pioglitazone hydrochloride and 500 mg metformin hydrochloride was administered after an overnight fasting of 10 hours with 240 ml of 20% glucose water. The OD dosage was compared to pioglitazone tablets 15 mg (Actos manufactured by Takeda Pharmaceuticals, USA) and metformin extended release 500 mg tablets (Glucophage XR tablets manufactured by Bristol-Myers Squibb, USA). There was a washout period of seven days. All subjects were on fast overnight for a period of 10 hours before commencement of dosing. Drinking water was not allowed from one hour pre-dosing to 10 hour post dosing. Uniform and low fat meals were provided to all the subjects.

The plasma pioglitazone and metformin concentrations were measured by high performance liquid chromatographic (HPLC) method using an ultraviolet (UV) detector. The results are provided below.

Pioglitazone: The OD formulation showed a Tmax of 2.9±0.1287 as compared to a Tmax of 3.02±0.3608 for the reference formulation, indicating that the test and reference formulations have substantially the same mean values.

The OD formulation gave a serum concentration time profile similar to the reference formulation. The peak serum concentration (Cmax) was comparable to that for the reference formulation, indicating a similar rate of absorption of pioglitazone. The total bioavailability of pioglitazone measured as area under the curve (AUC 0-∝) was also comparable to that of the reference tablets, indicating that the entire drug was released from the formulation and absorbed during its transit through gastrointestinal tract. These results are presented in Table 3.

TABLE 3
Piaglitazone Pharmacokinetic Data

| Parameters | Reference | Test |
|---|---|---|
| Cmax (ng/ml) | 743.588 ± 67.44 | 727.724 ± 118.21 |
| Tmax (hr) | 3.02 ± 0.3608 | 2.90 ± 0.1287 |
| AUC (0-∝) ng/ml · hr) | 5835.98 ± 1284.71 | 5554.94 ± 1232.29 |

Further, referring to Table 4, the extent of absorption for the test product was comparable to that for the reference product as indicated by the ratio of test to reference (T/R ratio). The 90% confidence intervals were found to be within the bioequivalence acceptance range of 80-120% for the untransformed data (as per Drug Controller General of India (DCGI) draft guidelines). The results are shown in Table 4.

TABLE 4
90% Confidence Intervals for Pioglitazone Pharmacokinetic data

| | Ratio (%) | 90% Confidence |
|---|---|---|
| Parameters | (Test/Reference) | Intervals |
| Cmax (ng/ml) | 97.75 | 90.97-104.53 |
| Aug (0-∝) (ng/ml · hr) | 94.79 | 86.92-102.66 |

Metformin: The OD formulation showed a Tmax of 3.88±0.8013 as compared to 3.58±0.8940 of reference formulation, indicating that test and reference formulations have nearly same mean values.

Referring to Table 5, the OD formulation made in accordance with Example 3 gave a serum concentration time profile similar to the reference formulation. The peak serum concentration (Cmax) was comparable to that for the reference formulation, indicating a similar rate of absorption of metformin hydrochloride. The total bioavailability of metformin measured as the area under the curve (AUC 0-∝) was also comparable to that of the reference tablets, indicating that the entire drug was released from the formulation and absorbed during its transit through gastrointestinal tract.

TABLE 5
Metformin Pharmacokinetic Data

| Parameters | Reference | Test |
|---|---|---|
| Cmax (ng/ml) | 633.227 ± 109.33 | 670.527 ± 116.392 |
| Tmax (hr) | 3.02 ± 0.3608 | 3.58 ± 0.8940 |
| AUC (0-∝) ng/ml · hr) | 4653.866 ± 1463.9 | 4380.234 ± 1110.44 |

Further, referring to Table 6, the extent of absorption for the test product was comparable to that for the reference product as indicated by the ratio of test to reference (T/R ratio). The 90% confidence intervals were found to be within the bioequivalence acceptance range of 80-120% for the untransformed data (as per DCG1 draft guidelines).

TABLE 6
90% Confidence Intervals for Metformin Pharmacokinetic data

| | Ratio (%) | 90% Confidence |
|---|---|---|
| Parameters | (Test/Reference) | Intervals |
| Cmax (ng/ml) | 107.24 | 96.23-118.25 |
| Aug (0-∝) (ng/ml · hr) | 96.43 | 84.07-108.79 |

While several particular forms of the invention have been illustrated and described, it will be apparent that various modifications and combinations of the invention detailed in the text can be made without departing from the spirit and scope of the invention. For example, a bilayered tablet comprising an extended-release biguanide in one layer and an immediate-release glitazone in another layer may be prepared of the example given below.

### EXAMPLE 4

Preparation of bilayered tablets:

| | INGREDIENTS | Mg/tablet |
|---|---|---|
| Metformin | Metformin Hydrochloride | 500 |
| layer | Microcrystalline Cellulose | 245 |
| | Sodium Carboxymethyl Cellulose | 150 |
| | Hydroxypropyl methylcellulose | 100 |
| | Magnesium Stearate | 5 |
| Seal Coat | Hydroxypropyl methylcellulose E5 | 15.6 |
| | Polyethylene glycol 4000 | 4.8 |
| | Titanium Dioxide | 2.4 |
| | Talc | 1.2 |
| Pioglitazone | Pioglitazone hydrochloride equiv. | 39.672 |
| layer | to pioglitazone (30 mg) | |
| | Lactose | 80 |
| | Hydroxypropyl cellulose | 2.4 |
| | Carboxymethyl cellulose calcium | 3.6 |
| | Magnesium stearate | 1.2 |
| | Purified water | q.s. |

Procedure:

1. Metformin hydrochloride was milled and mixed with microcrystalline cellulose and sodium carboxymethyl cellulose. The blend was sieved.

2. Hydroxypropyl methylcellulose was separately sifted and mixed with the blend of step 1 in a low shear mixer. The blend was then mixed with magnesium stearate and passed through roller compactor and then milled again to form granules.

3. Pioglitazone, lactose, hydroxypropyl cellulose and carboxymethylcellulose calcium were blended and granulated with purified water.

4. The wet mass of step 3 was granulated, dried and sifted.

5. The lubricated granules of metformin and pioglitazone were compressed into bilayer tablets using a rotary compression machine.

Further, it is contemplated that any single feature or any combination of optional features of the inventive variations described herein may be specifically excluded from the claimed invention and be so described as a negative limitation. Accordingly, it is not intended that the invention be limited, except as by the appended claims.

1. A solid pharmaceutical dosage form for oral administration, the dosage form comprising:
an extended release layer comprising a biguanide; and
an immediate release layer comprising a glitazone.

2. The dosage form of claim 1, wherein the biguanide comprises one or more of metformin, phenformin, and buformin.

3. The dosage form of claim 1, wherein the biguanide is metformin.

4. The dosage form of claim 1, wherein the glitazone comprises one or more of pioglitazone, rosiglitazone, troglitazone, ciglitazone and englitazone.

5. The dosage form of claim 4, wherein the glitazone is pioglitazone.

6. The dosage form of claim 1, wherein after oral administration the biguanide is released over a period of about 4 to about 36 hours.

7. The dosage form of claim 6, wherein the biguanide is released over a period of about 8 to about 24 hours.

8. (canceled)

9. (canceled)

10. (canceled)

11. The dosage form of claim 1, wherein the extended release layer comprises a matrix.

12. The dosage form of claim 11, wherein the matrix comprises a uniform mixture of the biguanide and one or more rate controlling polymers.

13. (canceled)

14. (canceled)

15. (canceled)

16. The dosage form of claim 1, wherein the biguanide is layered onto a pharmaceutically inert core or seed.

17. The dosage form of claim 16, wherein the inert core or seed is hydrosoluble or hydroinsoluble.

18. The dosage form of claim 1, wherein the immediate release outer layer further comprises film-forming polymers and optionally other pharmaceutically acceptable excipients.

19. (canceled)

20. The dosage form of claim 18, wherein the pharmaceutically acceptable excipients comprises one or more of plasticizers, opacifiers and colorants.

21. The dosage form of claim 1, further comprising one or more of sulfonylurea, insulin, alpha-glucosidase inhibitors, meglitinides, fibrates, statins, squalene synthesis inhibitors and angiotensin-converting enzyme inhibitors.

22. The dosage form of claim 1, further comprising a wetting agent in the immediate release layer, wherein the immediate release layer comprises the glitazone and the wetting agent in a weight ratio ranging from about 10:1 to about 1:25.

23. (canceled)

24. (canceled)

25. (canceled)

26. (canceled)

27. (canceled)

28. The dosage form of claim 1, wherein the extended release layer comprises a core and the immediate release layer covers at least a portion of the core.

29. The dosage form of claim 1, wherein the dosage form comprises a bilayered dosage form.

30. A process for preparing a solid, orally administered pharmaceutical dosage form of an extended release core of a biguanide and an immediate release layer of a glitazone, the process comprising:
a. dispersing the biguanide in a solid matrix to form a core having a surface; and
b. layering the immediate release layer of the glitazone on the surface of the core.

31. The process of claim 30, wherein layering the immediate release layer further comprises layering one or more wetting agents.

32. The process of claim 31, wherein the glitazone and the one or more wetting agents are present in the immediate release layer in a weight ratio ranging from about 10:1 to about 1:25.

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. The process of claim 30, further comprising placing a seal-coat over the core, wherein the seal-coat comprises hydrophilic polymers.

58. A process for preparing a bilayered, solid, orally administered pharmaceutical dosage form of a biguanide and a glitazone, the process comprising:
a. dispersing the biguanide in an extended release carrier base material;
b. separately dispersing the glitazone in an immediate release carrier base material; and
c. compressing the material of step a and step b to form bilayered tablet.

59. The process of claim 58, wherein the immediate release carrier base material further comprises one or more wetting agents before or after dispersing the glitazone.

60. The process of claim 59, wherein the glitazone and the one or more wetting agents are present in a weight ratio ranging from about 10:1 to about 1:25.

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. A method of treating non-insulin dependent diabetes mellitus in a patient in need thereof, the method comprising administering a solid, pharmaceutical dosage form of the combination of a biguanide and a glitazone, wherein the dosage form provides an extended-release of the biguanide and an immediate release of the glitazone.

87. The method of claim 86, wherein the biguanide comprises one or more of metformin, phenformin, and buformin.

88. (canceled)

89. The method of claim 86, wherein the glitazone comprises one or more of pioglitazone, rosiglitazone, troglitazone, ciglitazone and englitazone.

90. (canceled)

91. The method of claim 86, wherein after oral administration the biguanide is released over a period of about 4 to about 36 hours.

92. The method of claim 86, wherein the biguanide is released over a period of about 8 to about 24 hours.

93. (canceled)

94. The method of claim 86, wherein the dosage form further comprises one or more of sulfonylurea, insulin, alpha-glucosidase inhibitors, meglitinides, fibrates, statins, squalene synthesis inhibitors and angiotensin-converting enzyme inhibitors.
